(19) United States
(12) Patent Application Publication
(10) Pub. No.: US 2023/0149000 A1
(43) Pub. Date: May 18, 2023

(54) APPARATUS AND METHODS FOR ACQUISITION OF MICROBIOPSY TISSUE SAMPLES USING A LASER

(71) Applicant: BOARD OF REGENTS, THE UNIVERSITY OF TEXAS SYSTEM, Austin, TX (US)

(72) Inventors: Thomas E. MILNER, Austin, TX (US); Jason B. KING, Austin, TX (US); Nitesh KATTA, Austin, TX (US); James W. TUNNELL, Austin, TX (US)

(73) Assignee: BOARD OF REGENTS, THE UNIVERSITY OF TEXAS SYSTEM, Austin, TX (US)

(21) Appl. No.: 17/995,865

(22) PCT Filed: Apr. 9, 2021
(86) PCT No.: PCT/US21/26620

§ 371 Date: Oct. 10, 2022

Related U.S. Application Data
(60) Provisional application No. 63/008,201, filed on Apr. 10, 2020.

(51) Int. Cl. A61B 10/02; A61B 18/22

(52) U.S. Cl. CPC A61B 10/02; A61B 18/22; A61B 2018/2266; A61B 2018/2205; A61B 2018/00761

(57) ABSTRACT

Apparatus and methods for tissue excision. In certain aspects, the apparatus and methods include an annular converging laser beam. The annular converging laser beam can be directed to a surface of a tissue and displace a portion of the tissue in a single or multiple laser pulses. In particular aspects, the dosimetry of the laser beam (e.g. the beam shape, pulse energy and pulse duration) can be controlled to eject the portion of the tissue in a manner to reduce damage to the displaced tissue and the surrounding tissue.

### CROSS-REFERENCE TO RELATED APPLICATIONS

This application claims priority to U.S. Provisional Patent Application Ser. No. 63/008,201 filed Apr. 10, 2020, the entire contents of which are incorporated herein by reference.

This invention was made with government support under Grant no. T32 EB007507 awarded by the National Institutes of Health. The government has certain rights in the invention.

### BACKGROUND INFORMATION

Existing tissue excision apparatus and methods are limited by numerous operational issues that provide less than optimally desired results. The issues discussed herein are not intended to be an exhaustive list and are merely exemplary of some of the shortcomings associated with such apparatus and methods.

One significant issue with existing tissue excision apparatus and methods relates to their use in traditional biopsies and the volume of tissue excised. In particular, traditional biopsy techniques excise significantly more tissue than is actually used in many analyses. For example, only a small fraction (e.g. less than one percent) of excised tissue material is actually analyzed in a traditional biopsy using hematoxylin and eosin (H&E) stain.

Accordingly, the location from which the tissue is excised is subjected to more trauma than necessary to obtain a sample for such techniques. This can lead to a reluctance to take biopsies in cases where tissue must be surgically repaired (e.g. sutured, bandaged, etc.). Furthermore, sampling errors can be caused by typical biopsies that are acquired from an area much larger than intended. This can create issues in difficult to acquire locations that are near critical tissue structures (e.g. tissue conserving surgeries like brain surgery), where excising as little tissue as possible is desirable.

In addition, traditional laser tissue ablation and cutting is inefficient in removing tissue volumes. The laser cut has high precision (micron level resolution) but the ablation volume is low, and thus, the volumetric rate of tissue removal (volume per unit time) is relatively slow. In addition, laser tissue ablation and cutting of tissue results in the fragmentation of the excised tissue into small-sized submicron fragments.

Accordingly, systems and methods are desired that overcome these and other limitations associated with existing systems and methods.

### SUMMARY

In one aspect, embodiments disclosed herein comprise an annular converging laser beam to excise precise volumes of tissue with a single laser pulse. Embodiments of the present disclosure address limitations of existing tissue excision apparatus and methods including for example, scalpels, electrosurgery devices, ultrasonic aspirators, lasers and tumor excision devices.

Particular embodiments of the present disclosure include apparatus and methods to harvest sub-microliter (e.g. less than 1 mm³) tissue sections (“micro-volumes”) using a laser beam with an annular converging beam profile. During operation of exemplary embodiments, tissue can be harvested using a pulsed laser beam focused below the tissue surface. In one aspect, an annular converging beam profile ablates a portion of tissue, and a portion of the tissue in the center of the annulus is ejected. These ejected micro-volumes may be used for diagnosis, analysis, or tissue culture using a variety of techniques including, for example, routine histopathology, genetic profiling, flow cytometry, microscopy, proteomic assays, mass spectrometry, or primary tissue culture.

In comparison to typical laser ablation systems, embodiments disclosed herein can effectively increase the volumetric tissue removal rate per joule of incident pulsed laser energy. This can allow for smaller volumes of tissues for harvest that can be precisely located. In addition, exemplary embodiments can provide precise cutting that allows tissue excision near delicate structures as compared to traditional scalpels. In addition, thermal confinement with the apparatus and methods disclosed herein can result in less residual thermal damage compared to electrocautery devices. Furthermore, the potential for improved preservation of tissue structure and architecture compared to ultrasonic aspirator and traditional laser surgery methods can allow histological analysis. Additionally, disclosed embodiments may produce excised tissue fragments that are larger and easier to collect and process as compared to traditional laser ablation approaches.

Embodiments of the present disclosure may be used in a variety of applications where excising micro-volumes of tissue is desired. For example, certain embodiments can be used in the micro-excision of cells for primary derived cell culture, cells for primary derived xenograft models, and immune cells for immunotherapy approaches. Particular embodiments may also be used for micro-excision of hair follicles for transplant or micro-excision for hydrogel/cell transplant, for micro-medical device implant, for drug testing, for DNA analysis in forensics and/or for precision medicine applications.

Applications for this method of laser micro-volume excision include, for example, any circumstances when a current, conventional biopsy is used. Additional applications could include micro-biopsy to intraoperatively guide surgical resection of tumors, sample tumors for precision medicine, basic science studies of tumor heterogeneity, combined laser cutting and diagnosis, or tissue harvest for subsequent culture.

In addition, the methodology can be applied for surgical resection where large volumes of tissue must be removed. For example, a precise micro-volume excision can be used during surgery for tumor resection near delicate tissue structures. The laser micro-volume excision disclosed herein can allow for precise tissue removal with limited damage to surrounding structures. The rapid diagnosis of collected tissue sections could guide the surgeon in achieving clear tumor margins without removing more tissue than is necessary.

Laser tissue harvest methods disclosed herein could also be used to biopsy suspicious lesions, potentially without bleeding or the need for sutures. These micro-biopsies could be used to diagnose disease or determine mutation status to guide treatment of disease, such as determining BRAF mutation status for treatment of melanoma. In certain aspects, embodiments of the present disclosure may also be utilized for a surgical resection procedure similar to Mohs surgical procedures. In this approach, each tissue section that is removed via the micro-volume excision approach can be rapidly tested for detection of, for example, cancer so that the attending surgeon can determine if the tissue region just below the resected tissue is cancerous or not. Similar to a conventional Mohs procedure, the laser approach can be continued to resect tissue until all the cancer is removed. An advantage of this Mohs-like approach is that surgical procedure times can be reduced since each micro-volume can be quickly analyzed to determine if a clean margin exists.

In exemplary embodiments of the present disclosure the laser beam shape, wavelength, and dosimetry can be optimized for efficient harvest from tissues with different optical and mechanical properties. Such optimization can minimize thermal damage to the excised micro-volumes as well as remaining tissue from which the micro-excision is collected.

In certain embodiments the beam shaping optics and collection mechanism may be configured for laparoscopic, robotic or a handheld tool for tissue harvest. Such embodiments can be similar in form to an ultrasonic aspirator in which suction or a vacuum pressure is used to remove tissue sections. Alternative methods of beam shaping could also be used in certain implementations including use of a spatial light modulator, an annular aperture, phase plate, or a fiber bundle arranged in an annular shape. An optical fiber implementation will allow laparoscopic and endoscopic use of applications within body cavities.

Certain embodiments include a tissue excision apparatus comprising: a laser configured for emission of a beam of electromagnetic energy; optical components configured to modify the beam of electromagnetic energy to form an annular converging beam; and a control system configured to limit the emission of the beam of electromagnetic energy to a duration between 10 picoseconds and 10 milliseconds. In particular embodiments the laser is configured to emit the beam of electromagnetic energy at a wavelength between 1.2 μm and 2.5 μm. In some embodiments the emission of the beam of electromagnetic energy has a pulse energy between 10 mJ and 10 J, and in specific embodiments the laser is a Ho:YAG laser.

In certain embodiments the optical components comprise a non-spherical lens and/or an axicon. In particular embodiments the optical components comprise an optical element configured to collimate the beam, and in some embodiments the optical element is a lens. In some embodiments the optical components comprise a reflective collimator. In specific embodiments, the beam of electromagnetic energy is directed through an optical fiber, including for example a multimode fiber.

In certain embodiments the annular converging beam comprises a peripheral portion and a central portion, and in particular embodiments the central portion of the annular converging beam has lower electromagnetic energy fluence than the peripheral portion.

In certain embodiments, the tissue excision apparatus further comprises a cooling device configured to direct a coolant toward the beam of electromagnetic energy. In particular embodiments, the control system is configured to synchronize an application of the coolant with the emission of the beam of electromagnetic energy. In some embodiments, the cooling device comprises a solenoid valve. In specific embodiments, the cooling device comprises a nozzle. In specific the cooling device is configured to direct 1,1,1,2-Tetrafluoroethane or CO₂ toward the beam of electromagnetic energy.

Particular embodiments include a method of excising tissue, where the method comprises: directing a beam of electromagnetic energy to a surface of a tissue, where the beam of electromagnetic energy is an annular converging beam; and displacing a portion of the tissue. In some embodiments the beam is an annular converging cone-shaped beam. In specific embodiments the beam of electromagnetic energy is directed to the surface in a single pulse with a duration between 10 picoseconds and 10 milliseconds and in some embodiments the single pulse comprises a pulse energy of between 10 mJ and 10 J. In particular embodiments the electromagnetic energy has a wavelength between 1.2 μm and 2.5 μm.

In some embodiments displacing the portion of the tissue forms a void in the tissue, and in specific embodiments the void extends at least 100 μm from the surface of the tissue. In certain embodiments the void has a diameter between 100 μm and 1 mm, and in some embodiments the portion of the tissue has a volume between 0.003 mm³ and 0.3 mm³ or more particularly between 0.010 mm³ and 0.1 mm³.

Specific embodiments further comprise processing the portion of the tissue, and in certain embodiments processing the portion of the tissue comprises performing a diagnostic technique on the portion of the tissue. In particular embodiments processing the portion of the tissue comprises performing a tissue culture. In some embodiments processing the portion of the tissue comprises performing a histopathological examination. In specific embodiments processing the portion of the tissue comprises genetic profiling. In certain embodiments processing the portion of the tissue comprises flow cytometry. In particular embodiments processing the portion of the tissue comprises a proteomic assay. In some embodiments processing the portion of the tissue comprises mass spectrometry.

In certain embodiments the method comprises directing a coolant toward the surface of the tissue. In particular embodiments, directing the coolant toward the surface of the tissue is synchronized with directing the beam of electromagnetic energy to the surface of the tissue. In some embodiments of the method, the coolant is 1,1,1,2-Tetrafluoroethane or CO2.

In certain embodiments, the method further comprises applying an optical clearing technique to the surface of a tissue reduce tissue scattering. In particular embodiments, the optical clearing technique comprises applying a chemical agent to the surface of the tissue. In some embodiments, the chemical agent is glycerol. In specific embodiments, the optical clearing technique comprises applying mechanical pressure to the surface of the tissue.

In the present disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “approximately, “about” or “substantially” mean, in general, the stated value plus or minus 10%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed. In addition, a method that recites multiple steps does not require the steps be performed in the order recited.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 shows a schematic view of an apparatus according to an exemplary embodiment of the present disclosure during use.

FIG. 2 shows a perspective view of a portion of the apparatus of FIG. 1.

FIG. 3. shows a flowchart of aspects of a method according to an exemplary embodiment of the present disclosure during use.

FIG. 4 shows frames from a video of an exemplary embodiment of the present disclosure during use.

FIG. 5 shows optical coherence tomography (OCT) images of tissue after tissue excision according to an exemplary embodiment of the present disclosure.

FIG. 6 shows volumes of excised tissue for varying heights of the tissue surface in relation to the converging beam.

FIG. 7 shows OCT images of tissue after tissue excision according to the embodiments disclosed herein.

FIGS. 8-9 illustrate the relative fluence (energy per unit area measured) for simulated and measured beam shapes at different distances from the focus of the beam.

FIG. 10 illustrates the effects of pulse energy and beam focus with respect to successful displacement (e.g. ejection) of tissue from a tissue surface.

FIG. 11 shows the microbiopsy volumes collected versus the relative height of the tissue surface in relation to the converging beam.

FIG. 12 includes a graph showing crater depth versus relative tissue height and a graph showing crater width versus relative tissue height for the different pulse energies.

FIG. 13 includes a p-value table for the biopsy tissue portion volumes for the various pulse energy levels and tissue heights.

FIGS. 14-29 include images of the tissue after ablation, as well as the crater height, width, and biopsy volume for the five samples taken at each pulse energy level and tissue height.

FIGS. 30-36 illustrate simulated volume removal rates for annular and circular beams.

FIG. 37 illustrates results of a model exploring the effects of tissue scattering on thermal damage.

FIG. 38 illustrates strategies to achieve pre-conditioning of the tissue to extract material.

### DETAILED DESCRIPTION OF ILLUSTRATIVE EMBODIMENTS

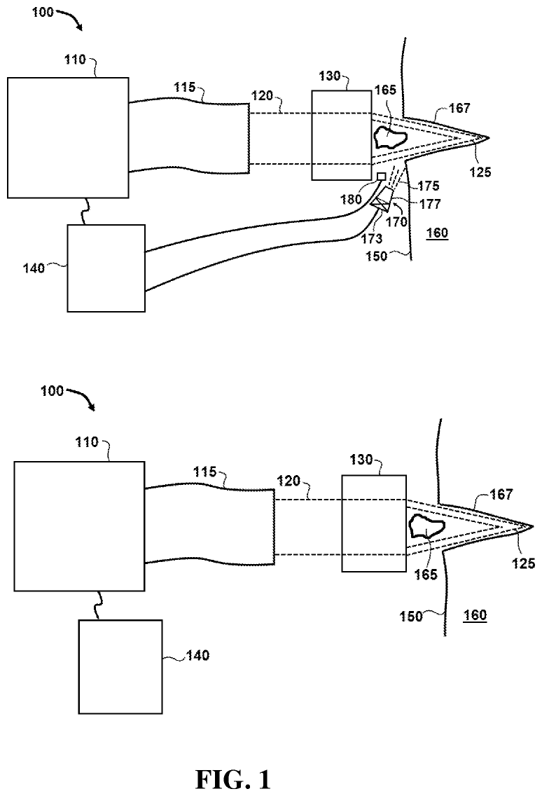

Exemplary embodiments of the present disclosure include apparatus and methods that utilize an annular converging laser beam to excise tissue in a single laser pulse. Referring initially to FIG. 1, a schematic of an apparatus 100 for tissue excision is shown during use. It is understood that the drawings disclosed herein are not to scale, and that some features may be enlarged or altered for purposes of clarity. In this embodiment, apparatus 100 comprises a laser 110 configured for emission of a beam 120 of electromagnetic energy. Apparatus 100 further comprises optical components 130 configured to modify beam 120 and a control system 140 configured to limit the duration of the emission of beam 120. As used herein, optical components include, but are not limited to, lenses, spatial light modulators, photomasks and other components configured to modify a beam of electromagnetic energy.

In particular, optical components 130 are configured to modify beam 120 to form an annular converging beam 125 which can be directed to a surface 150 of tissue 160. As used herein, an annular converging beam is interpreted as an annular beam with a cross-section that becomes smaller in the direction of beam propagation. Such beams include beams with both circular and non-circular cross-sections (e.g. triangular, square, or other polygonal-shaped cross-sections).

During operation, apparatus 100 can direct a single or multiple pulses from laser 110 to tissue 160 and eject a portion 165 of tissue 160. In some embodiments, beam 120 is directed from laser 110 through a multimode fiber 115. As explained further below, the operational parameters of apparatus 100 can be controlled such that portion 165 is excised from tissue 160 in a manner to minimize damage to portion 165 and surrounding tissue 160. For example, in certain embodiments, control system 140 controls laser 110 such that the duration of the pulse from laser 110 directed to tissue 160 is between 10 picoseconds and 1 millisecond. In addition, in certain embodiments laser 110 can be configured as a holmium yttrium-aluminum-garnet (Ho:YAG) laser, e.g. a solid-state laser that uses a holmium doping element in an yttrium-aluminum-garnet crystal. In particular embodiments, laser 110 is configured to emit energy at a wavelength between 1.2 μm and 2.5 μm, and the pulse energy for each pulse from laser 110 is between 10 mJ and 10 J. These and other parameters of apparatus 100 can be configured to harvest portion 165 in a manner to minimize damage to portion 165 and allow for effective further analysis of portion 165. In the embodiment shown, a void 167 is formed in tissue 160 in the area where portion 165 is ejected from tissue 160.

In certain embodiments, apparatus 100 may also comprise a cooling device 170 configured to direct coolant 175 toward beam 120 and tissue 160. Coolant 175 can be provided during operation of apparatus 100 to reduce the likelihood of thermal damage to portion 165 and/or tissue 160. In specific embodiments, cooling device 170 may be comprise a nozzle 177 and a solenoid valve 173 to provide the flow of coolant 175. Control system 140 can be used to control the flow of coolant 175 from cooling device 170. For example, a detector 180 can provide an electronic or wireless signal to control system 140 to control a flow of coolant 175 from cooling device 170.

In specific embodiments, detector 180 may be a photodetector that is configured to detect when a pulse from laser 110 is directed to tissue 160. In the embodiment shown in FIG. 1, detector 180 can send a signal to control system 140 to indicate that a pulse of beam 120 has been applied to tissue 160 and control system 140 can open solenoid valve 173 to provide for a flow of coolant 175 from nozzle 177 of cooling device 170. In particular embodiments, coolant 175 may be a 1,1,1,2-Tetrafluoroethane (e.g. R-134a), carbon dioxide (CO₂), water, air, or any suitable fluid (including liquid, gas, or liquid/gas mixtures).

In certain embodiments, control system 140 can control the flow of coolant 175 without the use of a detector such as detector 180. For example, control system 140 can comprise a timing control system to synchronize the pulse from laser 110 with the opening of solenoid 173. Based on the particular application (e.g. the configuration of apparatus 100, the type and location of tissue 160, etc.), control system 140 can control cooling device 170 to apply coolant 175 simultaneously with the pulse of beam 120, just before the pulse of beam 120 or just after the pulse of beam 120. In certain embodiments, the coolant can be applied in pulses with a duration of 10-300 milliseconds (ms).

Other embodiments may comprise additional or alternate features to reduce damage to portion 165 of tissue 160. For example, certain embodiments may incorporate optical clearing techniques to reduce tissue scattering, which can lead to damage within portion 165 (e.g. biopsied tissue). In particular embodiments, chemical optical clearing agents (e.g. glycerol) and/or mechanical pressure may be applied to the tissue to reduce tissue scattering and thus reduced damage to portion 165.

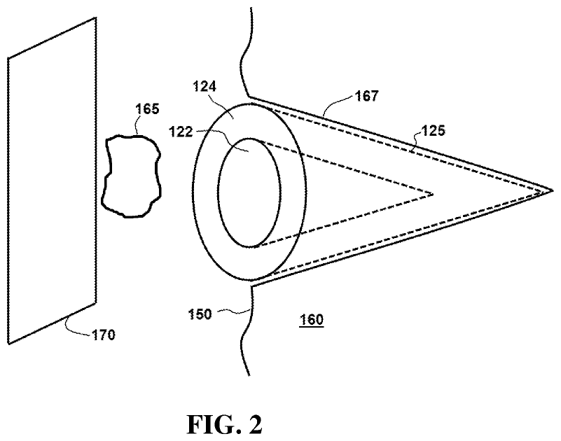

FIG. 2 illustrates a perspective schematic view of annular converging beam 125 and tissue 160. For purposes of clarity, other portions of apparatus 100 are not shown. While a cone-shaped beam is shown in the embodiment of FIG. 2, it is understood that other embodiments could include an annular converging beam with other shapes. For example, rather than the circular cross-section beam shown in FIG. 2, other embodiments may comprise a beam with cross-sections including triangular, square, or other polygonal-shaped cross-sections.

As shown in the figure, annular converging beam 125 comprises a central portion 122 and a peripheral portion 124. In exemplary embodiments, the electromagnetic energy of annular converging beam 125 is concentrated in peripheral portion 124, while central portion 122 has significantly lower electromagnetic energy in comparison to peripheral portion 124 (e.g. peripheral portion 124 has a higher fluence than central portion 122). The dosimetry of beam 125 (e.g. the beam shape, pulse energy and pulse duration) can be controlled to eject portion 165 from tissue 160 in a manner to reduce damage to portion 165 and tissue 160 surrounding void 167.

FIG. 2 shows portion 165 being directed toward a collection device 170. In the embodiment shown, collection device 170 is configured as a cover slip for use with a microscope slide to analyze portion 165. Other embodiments may comprise a collection device that incorporates features to assist in tissue collection, including for example, vacuum pressure. After portion 165 has been collected, it can be processed in any desired manner. In particular embodiments, portion 165 may be used for diagnosis, analysis, or tissue culture using a variety of techniques, e.g. routine histopathology, genetic profiling, flow cytometry, microscopy, proteomic assays, mass spectrometry, or primary tissue culture.

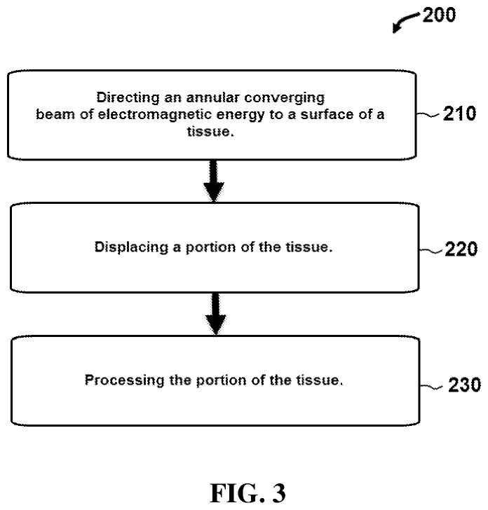

FIG. 3 provides a flowchart of a method 200 for excising tissue according to an exemplary embodiment. In this embodiment, method 200 comprises a first aspect 210 of directing an annular converging beam of electromagnetic energy to a surface of a tissue. In a second aspect 220, method 200 comprises displacing a portion of the tissue, and in a third aspect 230 comprises processing the portion of the tissue, including for example the diagnosis, analysis, or tissue culture techniques described above in the discussion of FIG. 2.

It is understood that other embodiments of methods for excising tissue with apparatus disclosed herein may comprise additional or fewer aspects than those disclosed in FIG. 3. For example, certain embodiments of use may not comprise analyzing the portion of the displaced tissue (which may be performed at a later time and/or by a different party than the party performing the aspects relating to directing the annular converging beam and displacing the portion of the tissue.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

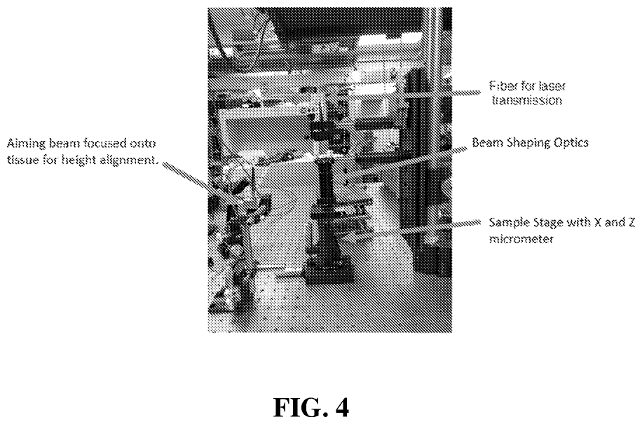

In one embodiment shown in FIG. 4, a benchtop implementation of an apparatus according to the present disclosure comprises: (1) a Ho:YAG (2.1 μm wavelength) laser source delivered through a 200 μm diameter optical fiber; (2) a 50 mm focal length aspheric zinc selenide (ZnSe) lens for collimating the light emitted from the fiber; (3) two fused silica axicons for shaping the beam into a collimated annular beam; and (4) a 25 mm focal length ZnSe lens for focusing the annular beam towards the tissue surface.

In this embodiment, the focused annular beam is absorbed by tissue water (absorption coefficient=27 cm⁻¹). Tissue in the beam region is ablated and pressure is generated in the surrounding tissue causing a micro-volumetric tissue section in the center of the annular beam to be ejected.

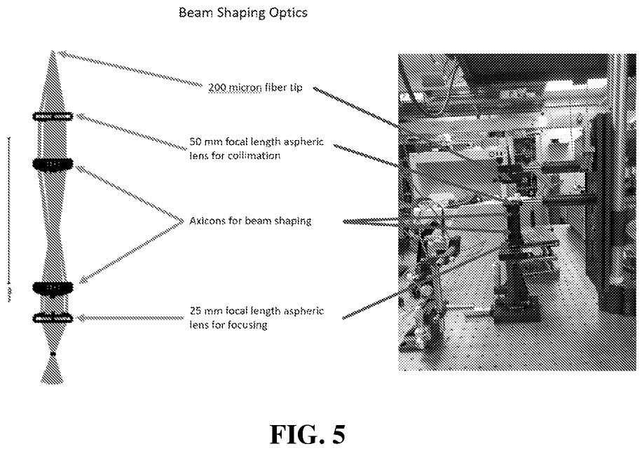

FIG. 5 shows a schematic diagram of optical components used in the apparatus shown in FIG. 4. As illustrated in the schematic, a 200 micron diameter fiber directs electromagnetic energy (e.g. light) to a 50 mm focal length aspheric lens for collimation and then to a pair of axicons for beam shaping. The electromagnetic energy is then directed to a 25 mm focal length aspheric lens for focusing.

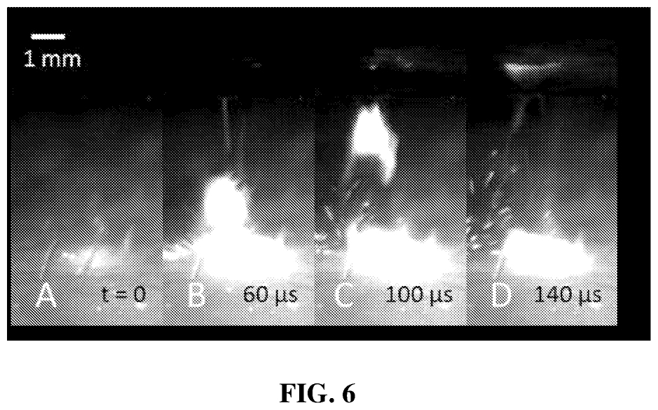

Referring now to FIG. 6, frames from a slow-motion video of an ablation process for a micro-biopsy performed using an apparatus as disclosed herein (including, for example, the example described above). The beginning of the laser pulse is shown in FIG. 6 Panel (A), and the tissue portion ejection is shown in FIG. 6 Panels (B) and (C). The specimen is shown adhered to the coverslip in FIG. 6 Panel (D). In this embodiment, the time duration from the beginning of the laser pulse emission to the tissue portion ejection was 60-80 μs (3-4 frames). The average speed of the ejected tissue portions was 38.3 m/s. In addition, the diameter of the void in the tissue (as measured by optical coherence tomography [OCT]) for three separate ablations were 460, 650, and 750 μm with corresponding depths of 280, 265, and 465 μm.

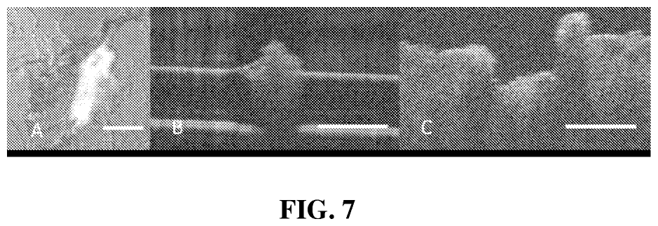

FIG. 7 shows ultrasound images of tissue after tissue excision according to the embodiments disclosed herein. Specifically, FIG. 7 shows a B-scan optical coherence tomography (OCT) image through the center of a residual void in the tissue in panel (A) and a B-Scan OCT image through center of microbiopsy tissue portion in panel (B). In addition, FIG. 7 shows an en-face view of collected microbiopsy in panel (C). In the images shown in FIG. 5, the scale bar is equal to 500 μm. The corresponding volumes of collected microbiopsies shown in FIG. 5 were approximately 0.030, 0.027, and 0.014 mm³ in panels (A), (B) and (C) respectively.

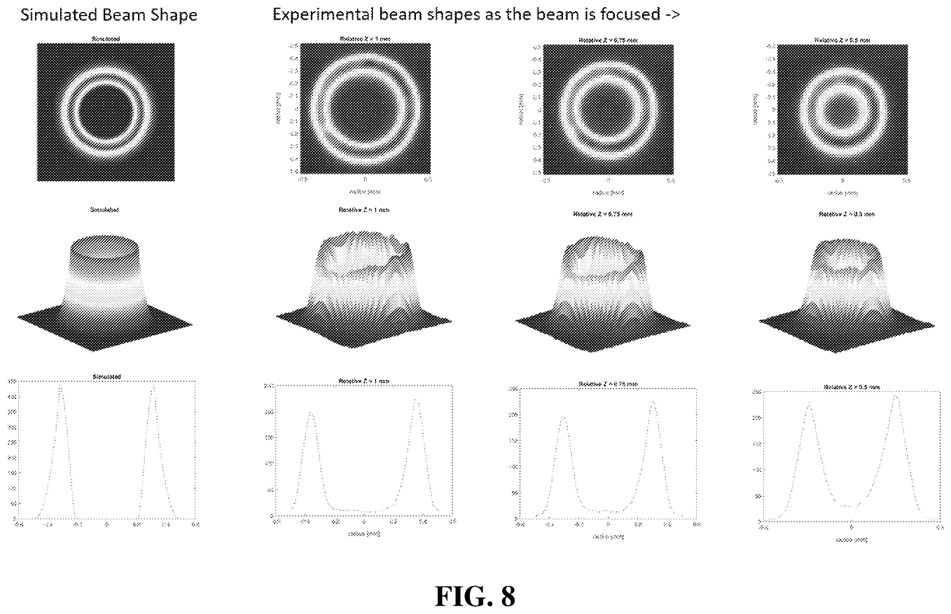

FIG. 8 illustrates the relative fluence (energy per unit area measured) for simulated and measured beam shapes. For the simulated beam, the values are in J/cm² and are from a simulated 1 J pulse based on the parameters described in the measured results below. In the simulated beam, the fluence is maximized at between 400 and 450 J/cm² between 0.2 mm and 0.5 mm from the center of the beam.

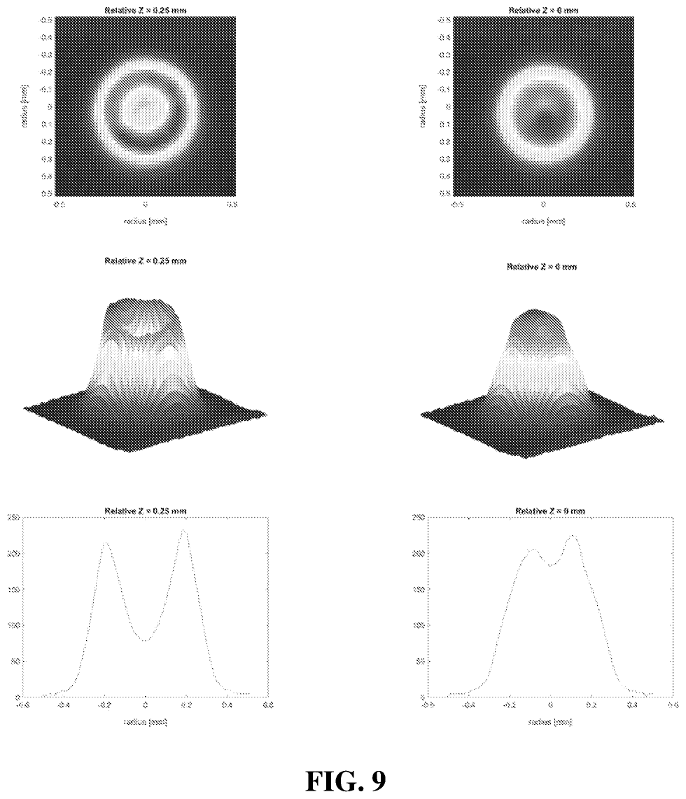

To obtain the measured data for the experimental beam shapes, a capillary (100 μm thick by 1.75 mm wide) with a thin layer of water was placed at different distances from the focal point of a converging annular laser beam. In FIGS. 8 and 9, the capillary tube was placed at different distances from the focal point of a beam where z=0 would be closest to the focus of the beam and 1.0 mm would be 1 mm further from the focus than z=0 (in contrast to other results discussed herein, where a higher value was closer to the focus of the beam).

The laser beam was pulsed to illuminate the water in the flat capillary tube and the heat generated in the water was measured with an infrared (IR) camera. The results along the y-axis for the measured values are relative and would be scaled based on the total pulse energy. As shown in the measured results, the fluence is greater in the peripheral portion of the laser beam and reduced in the central portion of the laser beam.

FIG. 9 illustrates the measured results as the flat capillary tube was moved closer to the focus of the beam (z=0 and 0.25 mm). As shown in FIG. 9 the annular shape is not as well defined as it gets closer to the focus, which can the limit to the smallest beam (and corresponding biopsy volumes) that can be obtained. In the apparatus used to obtain this data, the diameter of the optical fiber was 200 μm. It is expected that a smaller fiber (e.g. 50 μm diameter) may allow a smaller annular shape and corresponding biopsy volume.

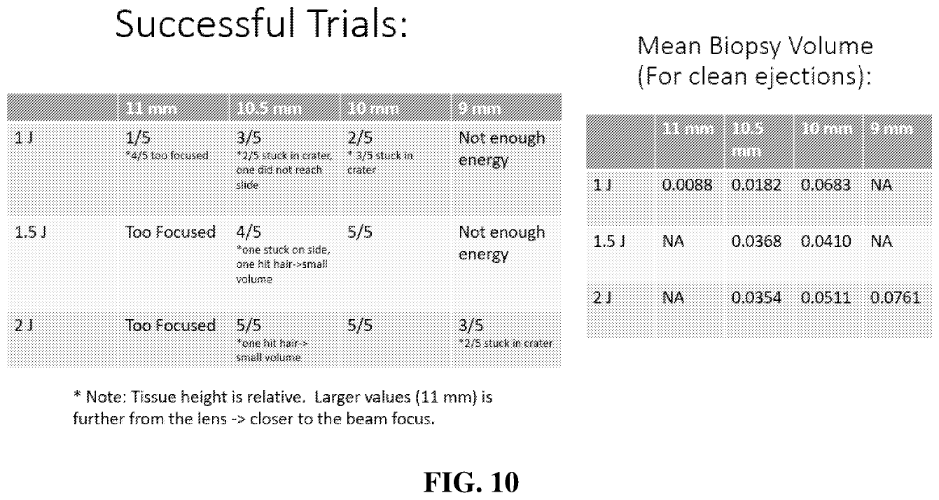

FIG. 10 illustrates the effects of pulse energy and beam focus with respect to successful displacement (e.g. ejection) of tissue from a tissue surface. In the data shown, the laser pulse energy levels were varied between 1 J, 1.5 J and 2 J, and the distance from the lens was varied from 11 mm, 10.5 mm, 10 mm and 9 mm. The test was repeated five times at each energy level and lens distance. As shown in FIG. 10, the pulse energy level and lens distance (e.g. which is related to the distance from the beam focus, and therefore the beam diameter) can significantly affect the ability to successfully displace the biopsy sample portion from the tissue surface.

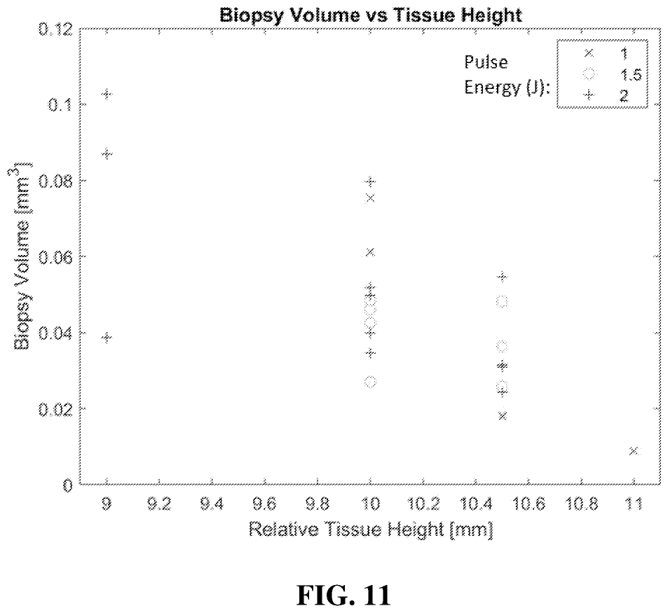

Referring now to FIG. 11, recent experiments have shown that the size of excised tissue and void left in the tissue can be altered by changing the beam diameter and pulse energy. Volumes of excised tissue ranged from 0.009 mm³ (pulse energy=1 J, crater diameter=334 μm) to 0.103 mm³ (pulse energy=2 J, crater diameter=640 μm). FIG. 11 shows the microbiopsy volumes collected versus the relative height of the tissue surface in relation to the converging beam, where 11 mm is closest to the focus of the beam and 9 mm is furthest from the focus of the beam.

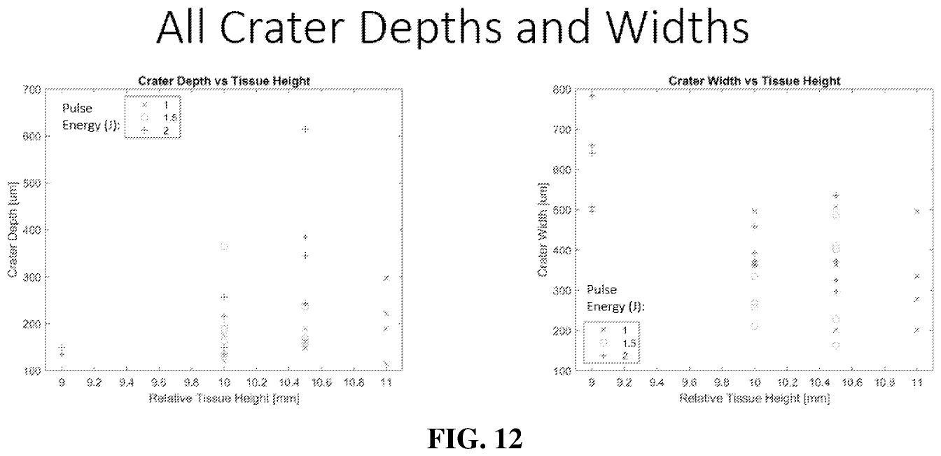

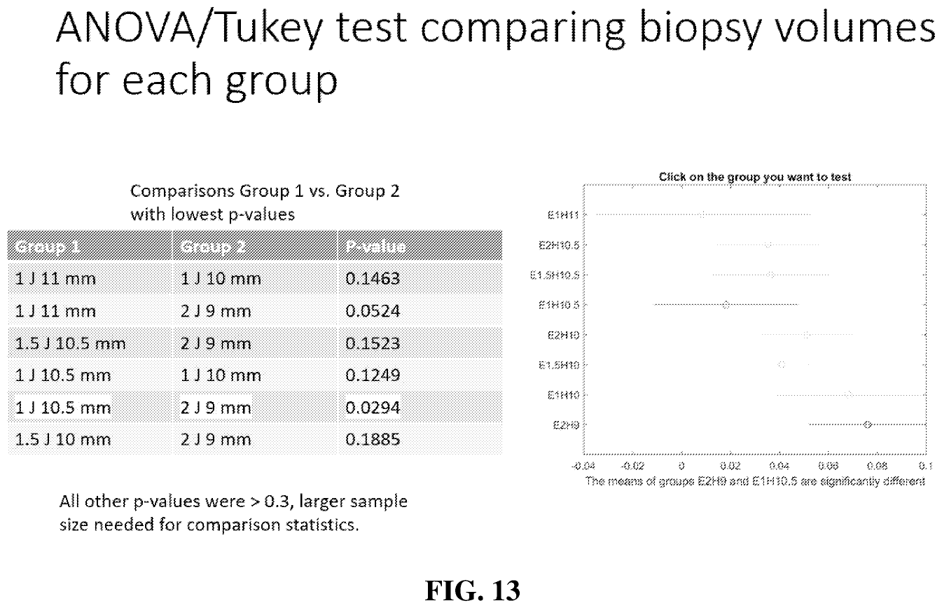

FIG. 12 includes a graph showing crater (e.g. the void formed when the pulsed laser ablates the tissue) depth versus relative tissue height and a graph showing crater width versus relative tissue height for the different pulse energies. FIG. 13 includes a table of the probability value (p-value) for the biopsy tissue portion volumes for the various pulse energy levels and tissue heights.

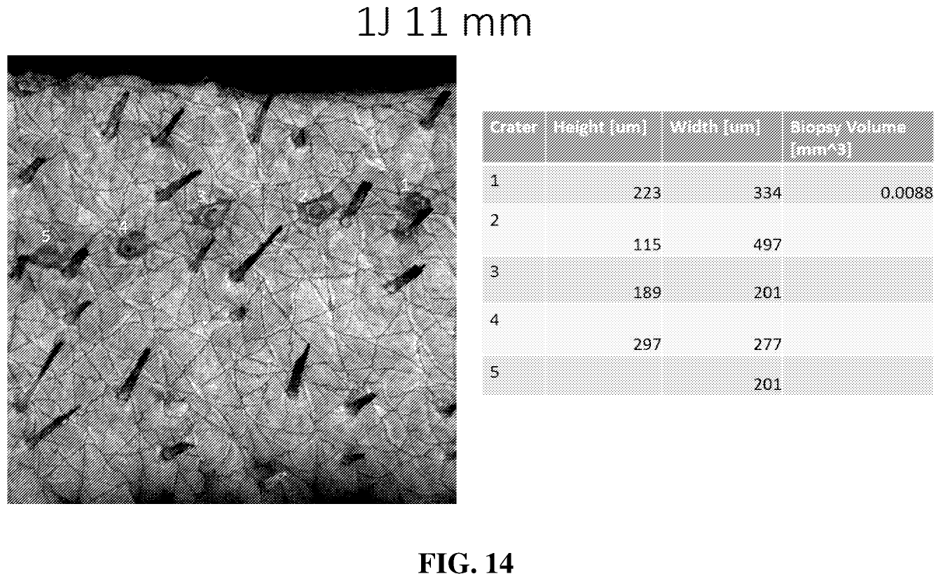

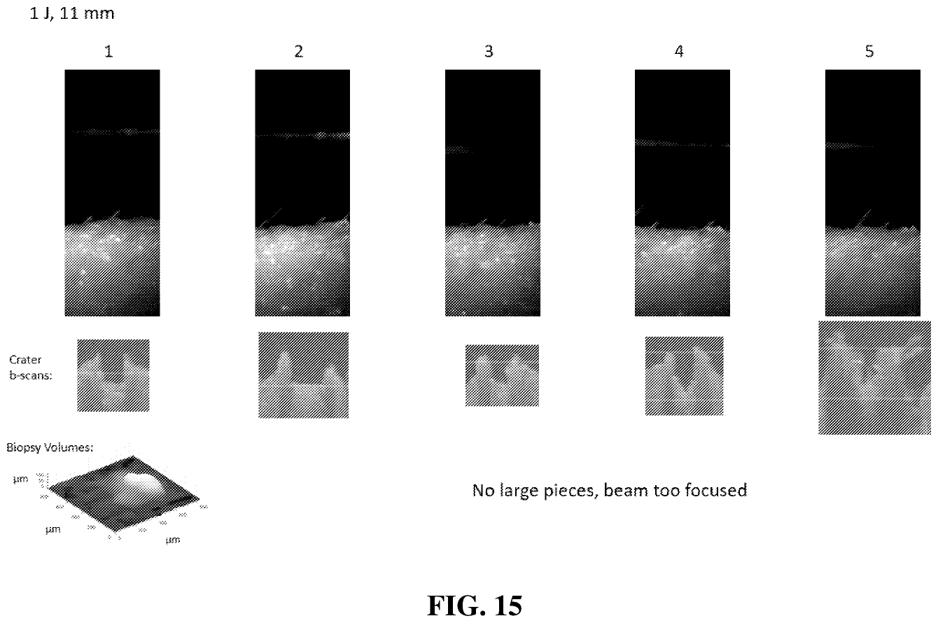

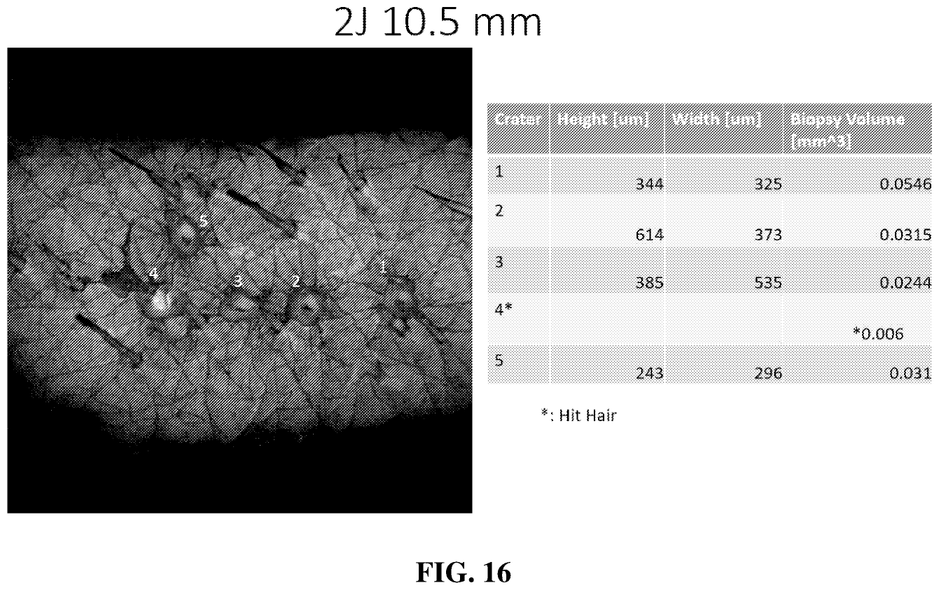

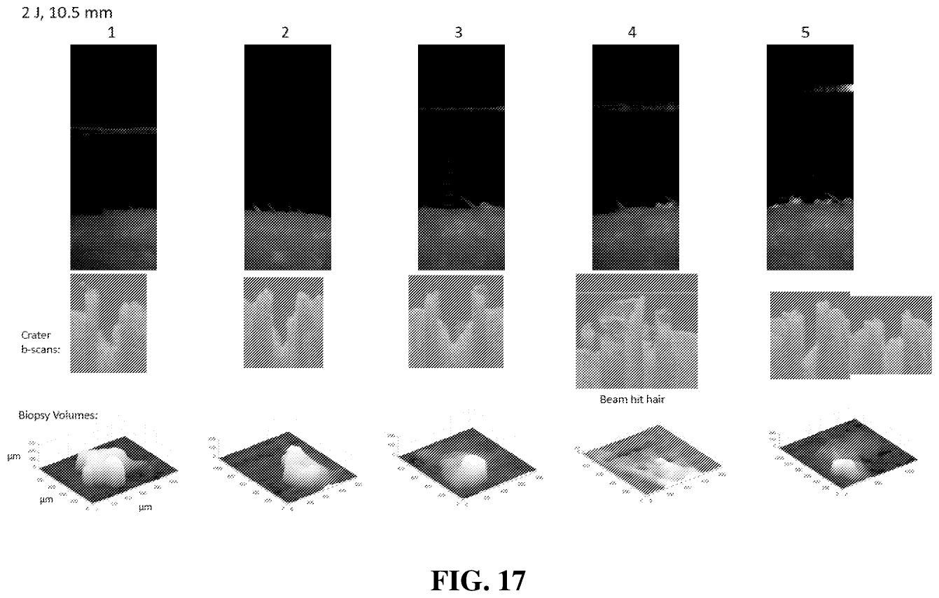

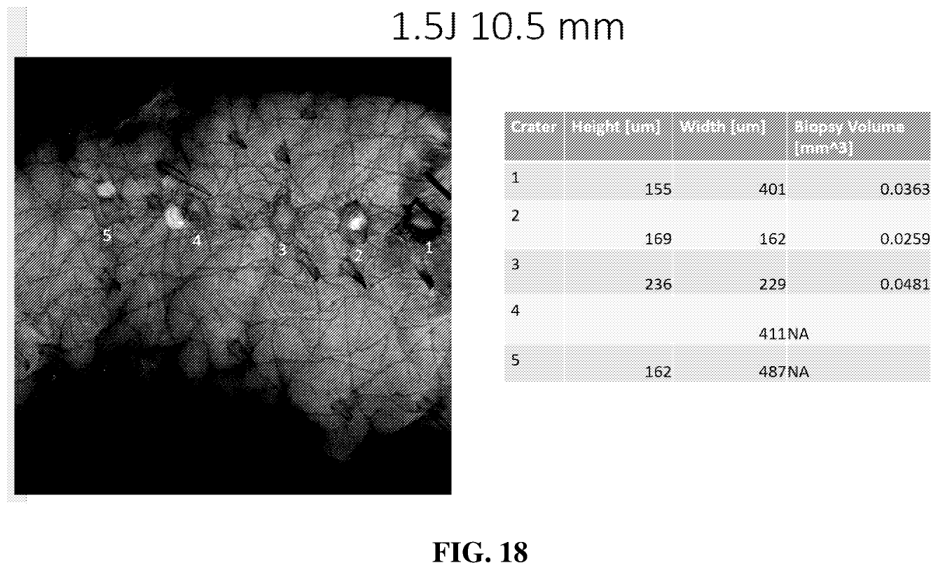

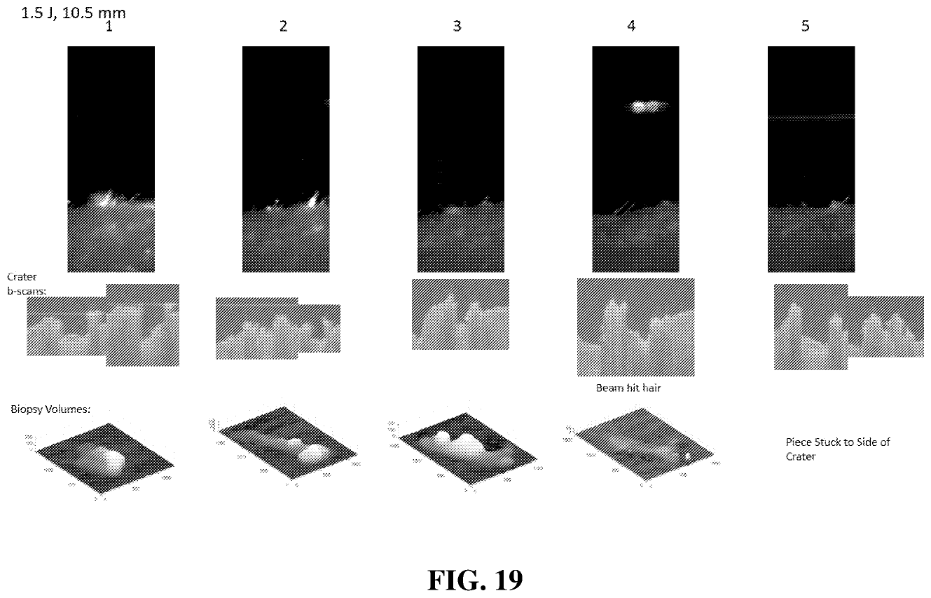

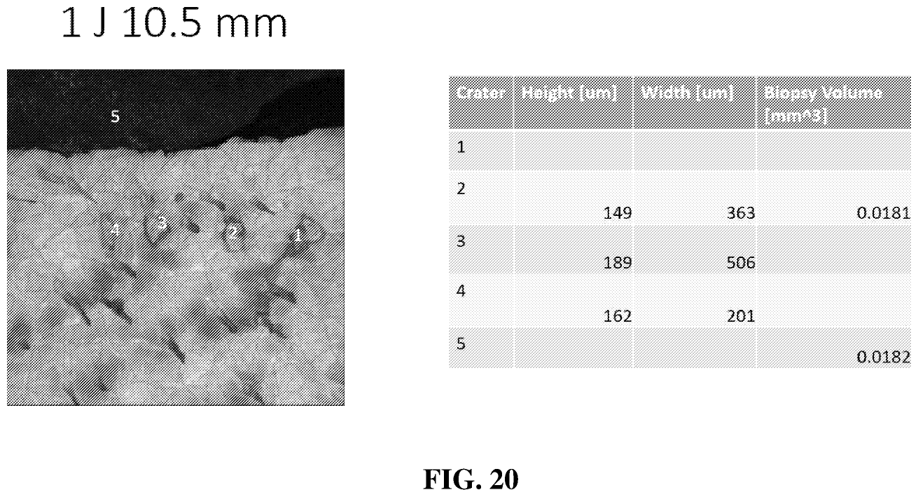

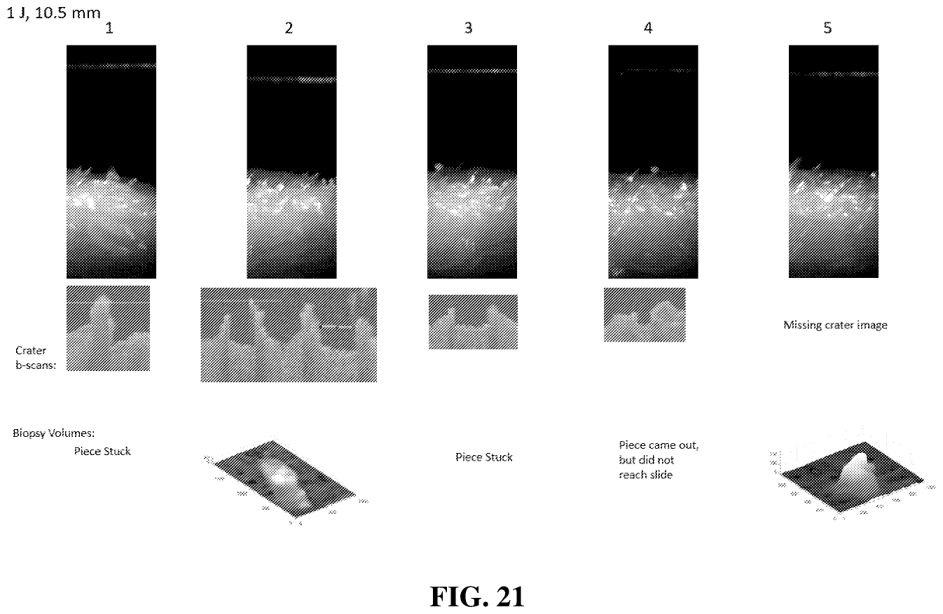

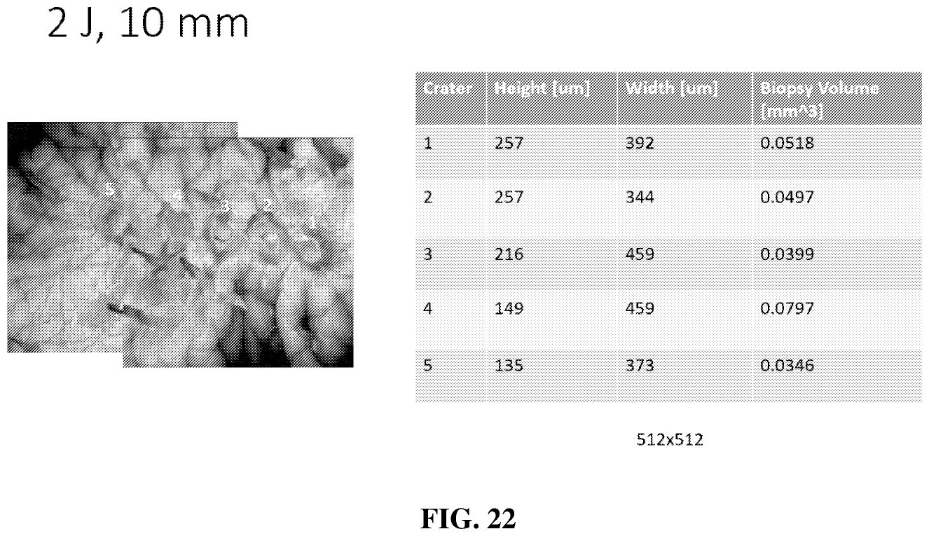

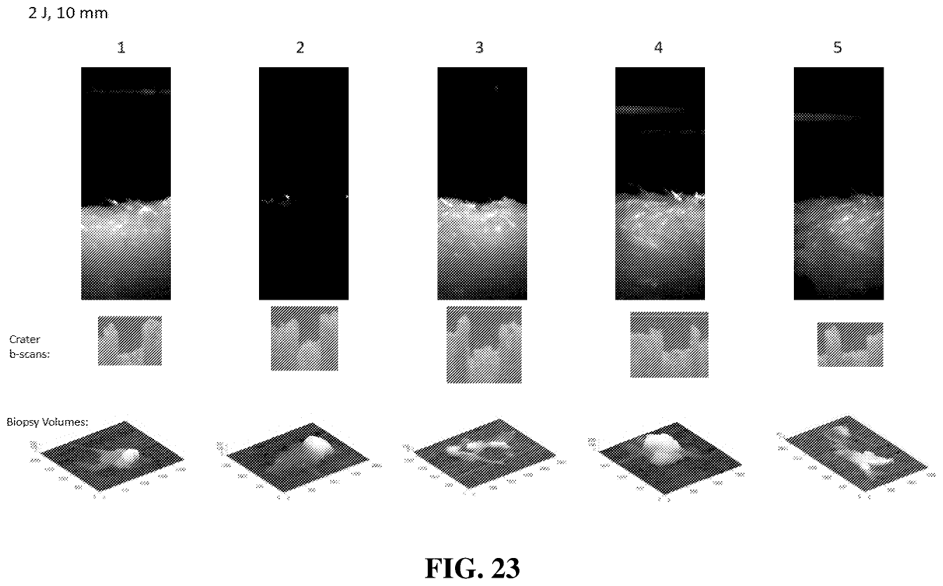

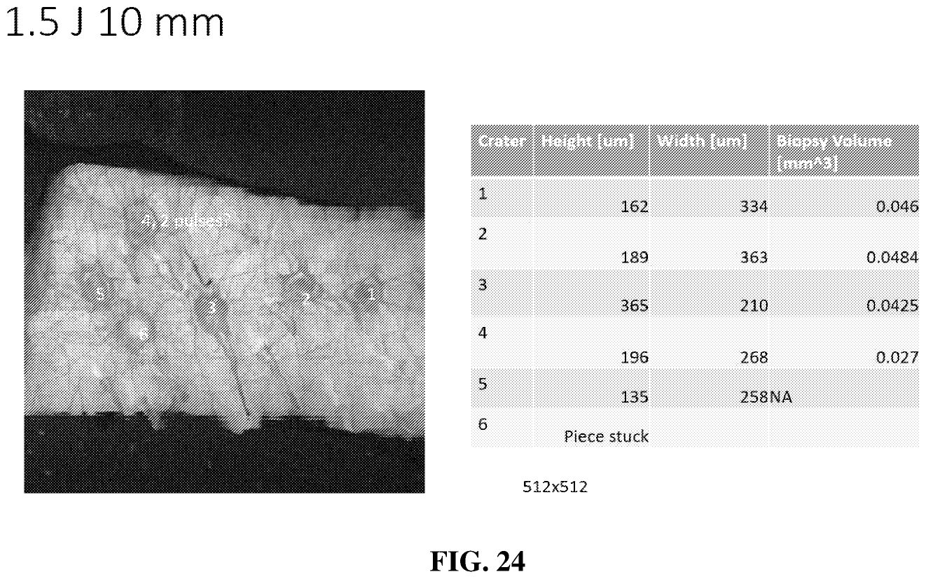

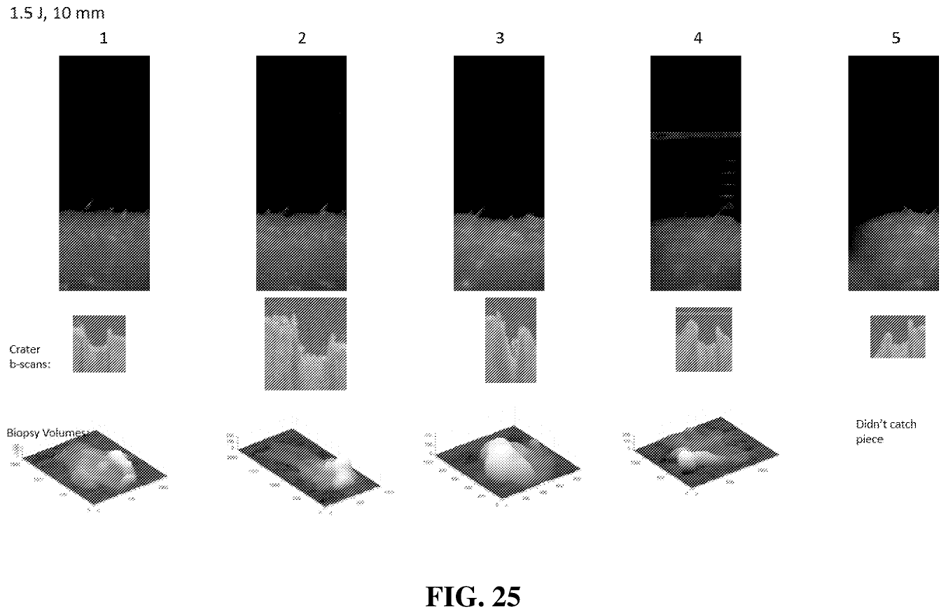

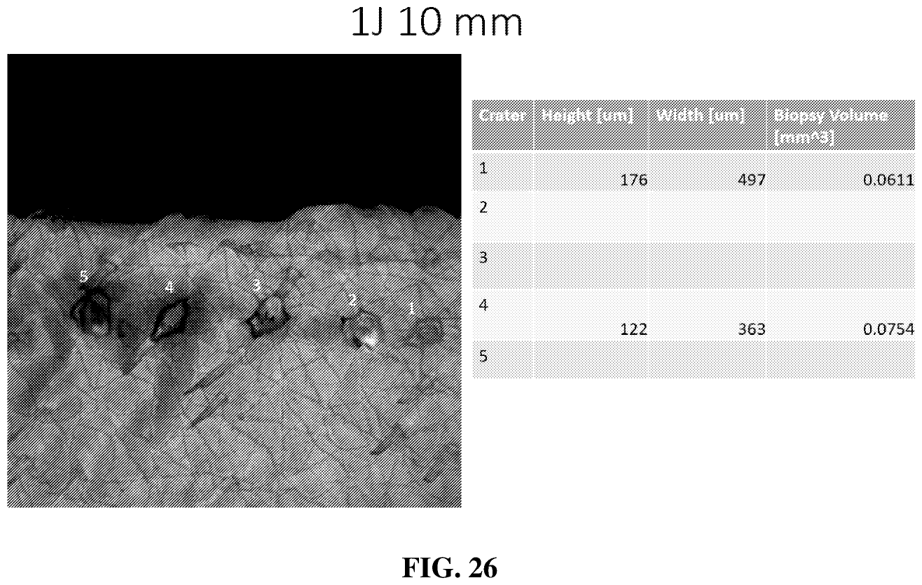

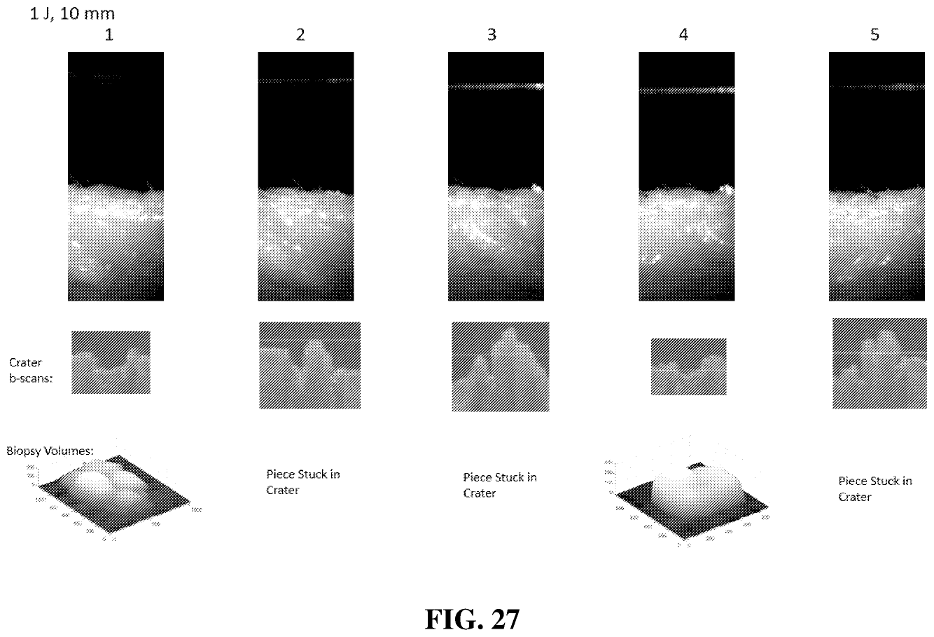

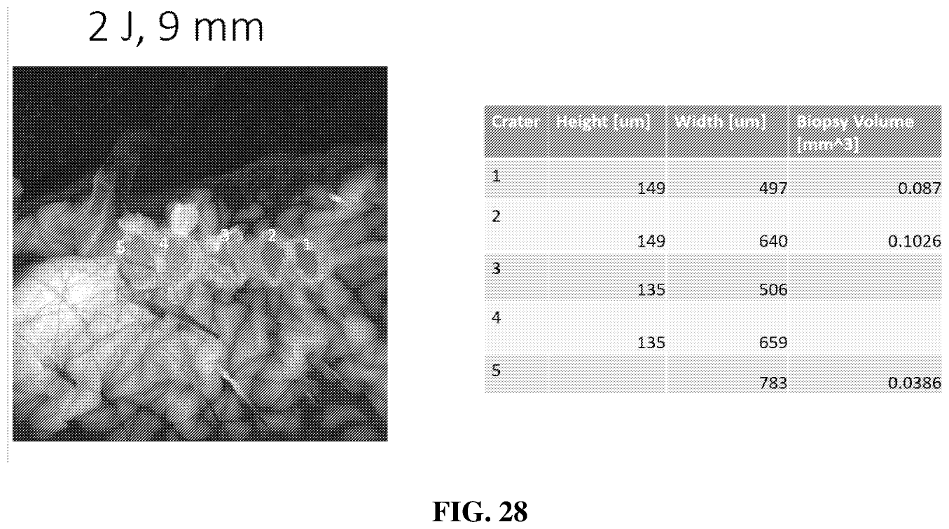

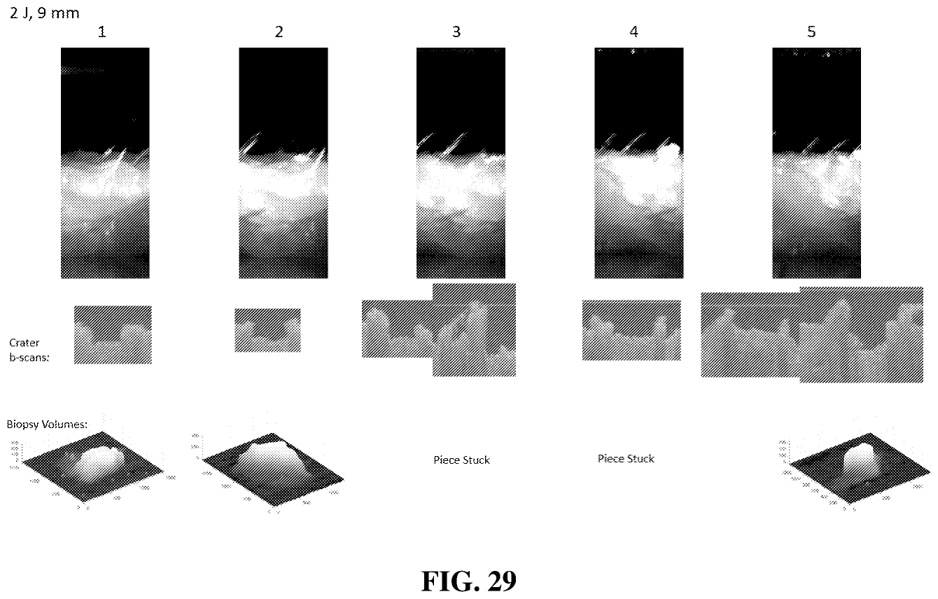

FIGS. 14-29 include images of the tissue after ablation, as well as the crater height, width, and biopsy volume for the five samples taken at each pulse energy level and tissue height.

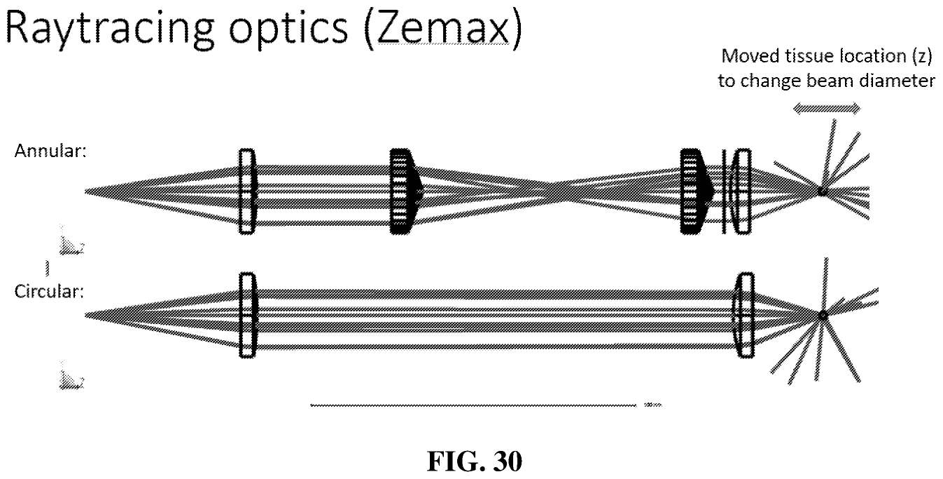

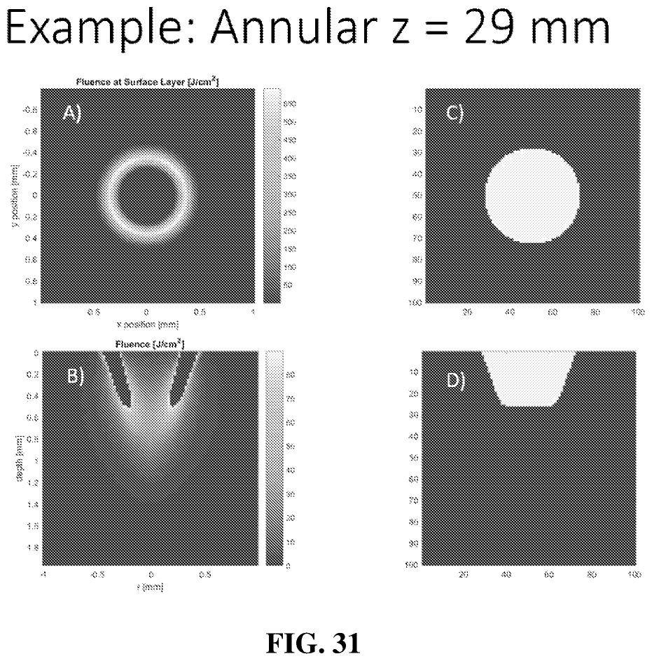

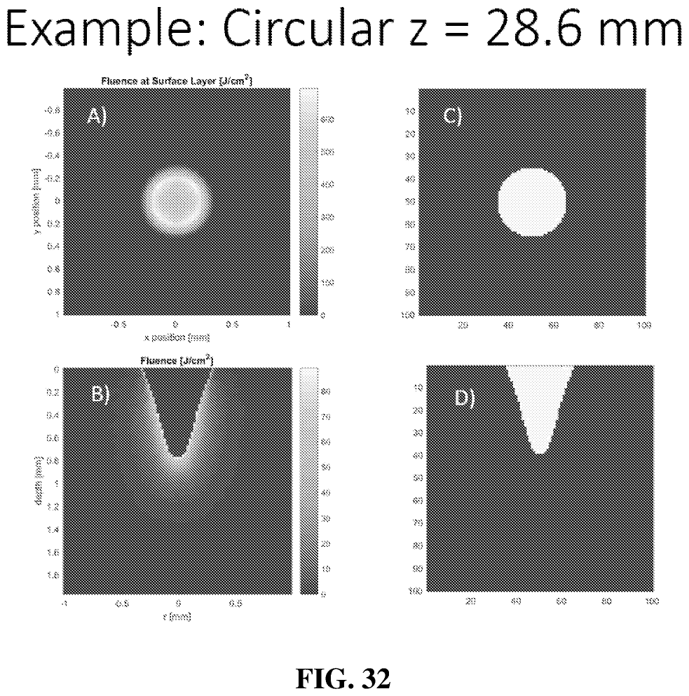

FIGS. 30-36 illustrate simulated volume removal rates of soft tissue for annular and circular beams, where scattering anisotropy factor g=0.9, scattering length lₛ=170 um, absorption coefficient μₐ=30 cm-1, pulse energy at tissue=1 J. Tissue locations were adjusted resulting in different beam diameters at the tissue surface, where annular z from lens=29.2, 29.0, 28.8, and 28.6 mm and circular z from lens=29.0, 28.8, 28.6, 28.4, 28.2, and 28.0 mm. FIG. 30 illustrates a schematic of the annular and circular simulations, while FIGS. 31-32 illustrate example calculations for annular and circular beams, respectively.

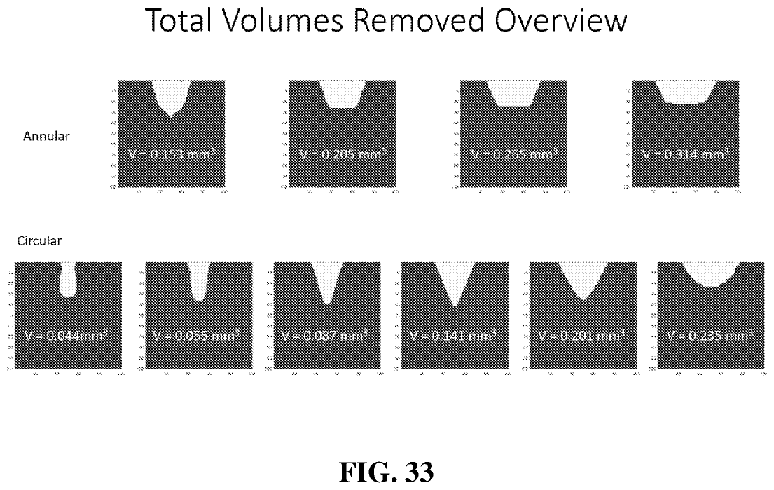

In FIG. 31, example calculations for an annular beam illustrate: (A) Fluence of the beam at the tissue surface and (B) Cross section fluence of the beam as it penetrated into the tissue, areas with fluence greater than the ablation threshold are removed (dark blue). The tissue volume inside the radius of the ablated annulus are assumed to be ejected. Panels (C) and (D) illustrate masks showing the tissue volume removed from the same view as in (A) and (B). The simulations indicate a total volume of 0.205 mm³ tissue was removed with a microbiopsy volume of 0.083 mm³. FIG. 32 illustrates example calculations for a circular beam: (A) Fluence of the beam at the tissue surface. (B) Cross section fluence of the beam as it penetrated into the tissue, areas with fluence greater than the ablation threshold are removed (dark blue). Panels (C) and (D) illustrate masks showing the tissue volume removed from the same view as in (A) and (B). The simulations indicate a total volume of 0.087 mm³ tissue was removed. FIG. 33 illustrates total volumes removed for annular and circular simulations.

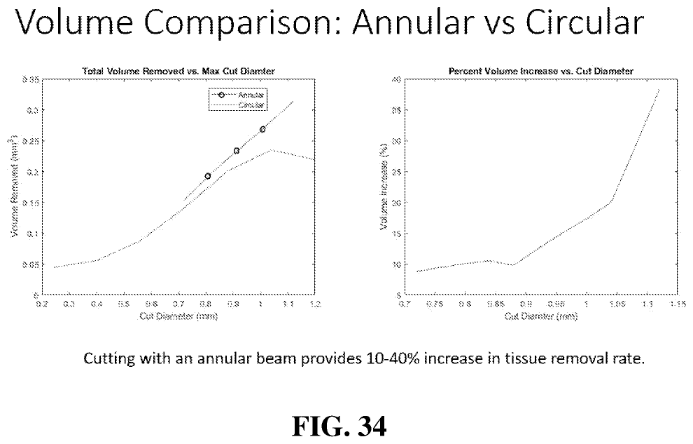

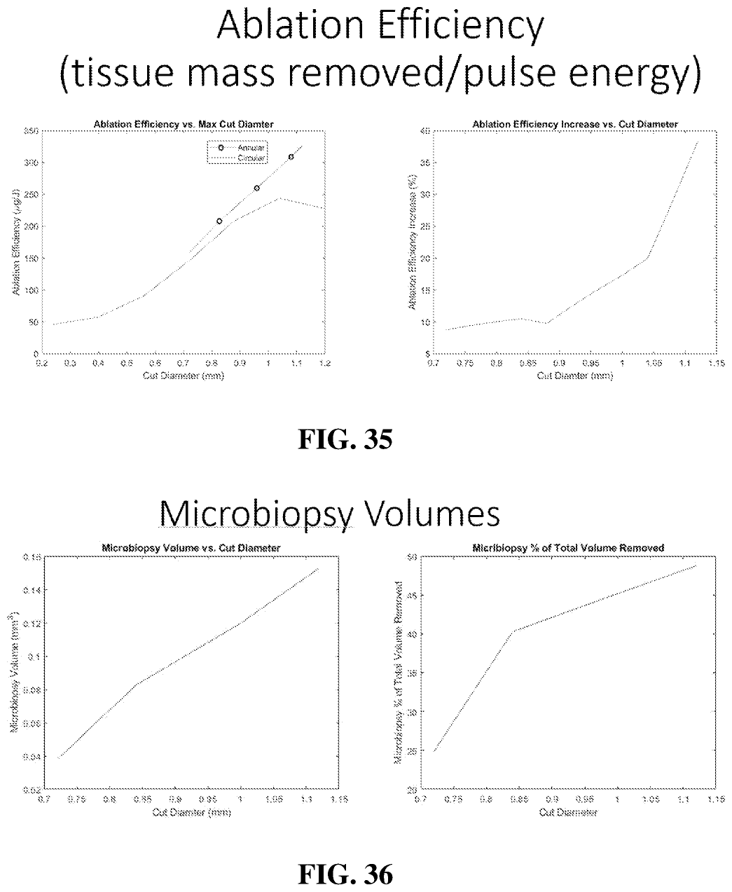

FIG. 34 illustrates a simulated volume comparison of annular versus circular beams. As shown in the graphs, the annular beam provides a 10-40% increase in tissue removal rate. FIG. 35 illustrates a simulation of ablation efficiency, e.g. tissue mass removed/pulse energy. FIG. 36 illustrates simulated microbiopsy volumes and microbiopsy percentage of total volume versus cut diameter, which were comparable to experimental results.

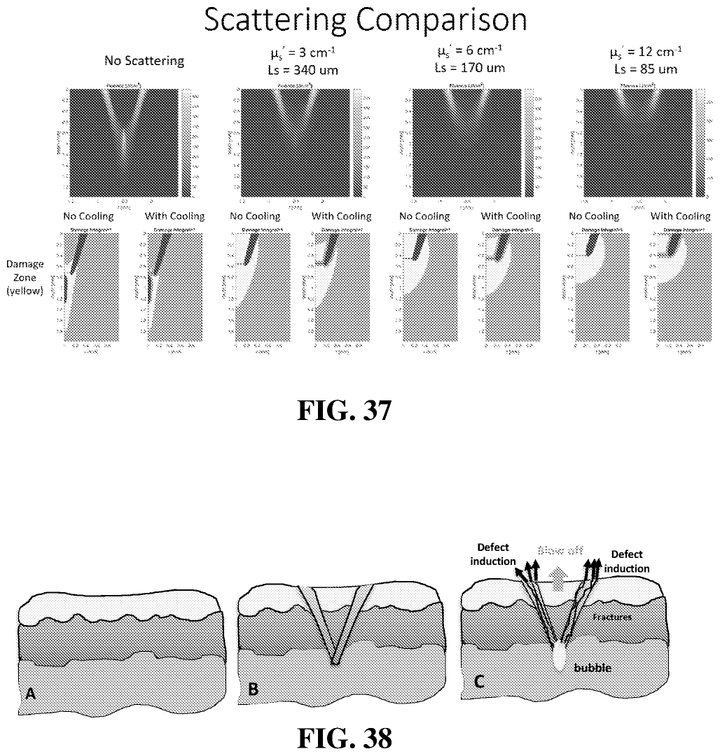

FIG. 37 illustrates results of a model exploring the effects of tissue scattering on thermal damage. The damage zone appears as yellow (or light gray in grayscale) in the model results shown. In the model, Raytracing was used to find fluence through tissue with the following parameters: 1 J pulse energy at tissue surface (˜1.5 J from laser); Absorption coefficient: μₐ=30 cm⁻¹; Normal scattering coefficient: μₛ′=6 cm⁻¹; Additional scattering coefficients: μₛ′=12 cm⁻¹, μₛ′=3 cm⁻¹, no scattering; Ablation threshold=92.6 J/cm\hat{ }2; Set everything >100 C not ablated to 100 C; Convective (air or cryogen spray cooling) boundary around biopsy and crater; Calculated Arrhenius damage integral, value >1 indicates thermal damage. From the scattering model, it can be concluded that thermal damage is primarily due to light scattering into the center of the annulus and heating the microbiopsy. In addition, the combination of reducing scattering coefficient and cryogen spray cooling is expected to greatly increase integrity of harvested microbiopsies, and optical clearing methods to reduce tissue scattering, including optical clearing agents (e.g. glycerol) and mechanical pressure.

Certain embodiments of the present disclosure may also incorporate laser pre-conditioning for enhanced ablation. For example, the apparatus can be configured to include a pre-conditioning light pulse that can enhance the micro-biopsy. The pre-conditioning pulse can be applied through a distinct optical path (different from the tissue excision laser path) or combined in the same path. Examples of such techniques have been previously described PCT Patent Publication WO 2020/231975, the entire contents of which are incorporated herein by reference.

The application of a pre-conditioning pulse can enhance the laser microdissection process. More specifically, the pre-conditioning pulse, can be applied to provide a more consistent and controlled tissue harvest. The preconditioning pulse can be applied to reduce the shear modulus of the targeted tissue to improve the laser micro-dissection process.

Complementary to the tissue removal aspects disclosed herein is a defect-induction step that produces a spatially patterned temperature increase. Spatial-patterning of the defect-inducing step allows microcrack expansion and fracture propagation to be spatially confined to selected regions in the target material. For example, utilizing axicons for the defect-inducing step can be configured to generate a surface-confined conical region so that fracture propagation and material blow-off is spatially controlled and limited to a conical region (FIG. 38). The axicon configuration combined other tissue removal aspects disclosed herein (e.g. apparatus and methods utilizing an annular beam) can provide for material blow-off with minimal thermal modification in a relatively large tissue volume. This configuration can be useful for tissue harvesting or micro-biopsy so that a diagnostic screening approach can be applied characterize harvested tissue.

FIG. 38 illustrates strategies to achieve pre-conditioning of the tissue to extract material. The figure illustrates strategies to achieve a spatial patterning procedure to extract material (FIG. 38 Step A). The method involves modification of tissue with a conditioning pulse before/during short pulsed laser irradiation to create a bubble (FIG. 38 Step B). Modulus gradient is achieved in an axicon shape (FIG. 38 Step B) with the conditioning pulse that channels the fractures aiding the failure of the material along the axicon defect induction channels resulting in a blow off event (green arrow) (FIG. 38 Step C).

### REFERENCES

The following references are incorporated herein by reference:
Park, S. et al. A novel microactuator for microbiopsy in capsular endoscopes. J. Micromechanics Microengineering 18, (2008).
Lei, B. U. W. et al. Absorbent Microbiopsy Sampling and RNA Extraction for Minimally Invasive, Simultaneous Blood and Skin Analysis. J. Vis. Exp. 1-8 (2019) doi:10.3791/58614.
Navone, R. et al. Oral potentially malignant lesions: First-level micro-histological diagnosis from tissue fragments sampled in liquid-based diagnostic cytology. J. Oral Pathol. Med. 37, 358-363 (2008).
Vaskova, M. et al. Cytometric analysis of cell suspension generated by cavitron ultrasonic surgical aspirator in pediatric brain tumors. J. Neurooncol. 143, 15-25 (2019).
Zadeh, G. et al. Diagnostic implications of histological analysis of neurosurgical aspirate in addition to routine resections. Neuropathology 32, 44-50 (2012).
Behnan, J. et al. Ultrasonic Surgical Aspirate is a Reliable Source for Culturing Glioblastoma Stem Cells. Sci. Rep. 6, 1-15 (2016).
Hollon, T. C. et al. Near Real-Time Intraoperative Brain Tumor Diagnosis Using Stimulated Raman Histology and Deep Neural Networks. Neurosurgery 66, (2019).
Elfer, K. N. et al. DRAQ5 and eosin (‘D&E’) as an analog to hematoxylin and eosin for rapid fluorescence histology of fresh tissues. PLoS One 11, 1-18 (2016).
Eberlin, L. S. et al. Pancreatic Cancer Surgical Resection Margins: Molecular Assessment by Mass Spectrometry Imaging. 1-21 (2016) doi:10.1371/journal.pmed.1002108.
Lin, Lynlee L et al. “Microbiopsy engineered for minimally invasive and suture-free sub-millimetre skin sampling.” F1000Research vol. 2 120. 2 May 2013, doi:10.12688/f1000research.2-120.v2
Suter, V. G. A., et al. A randomized controlled clinical and histopathological trial comparing excisional biopsies of oral fibrous hyperplasias using CO2 and Er:YAG laser. Lasers Med Sci 32, 573-581 (2017). https://doi.org/10.1007/s10103-017-2151-8.
Monteiro, L. et al. “A histological evaluation of the surgical margins from human oral fibrous-epithelial lesions excised with CO2 laser, Diode laser, Er:YAG laser, Nd:YAG laser, electrosurgical scalpel and cold scalpel.” Medicina oral, patologia oral y cirugia bucal vol. 24, 2 e271-e280. 1 Mar. 2019, doi:10.4317/medoral.22819.
Gultepe, E. et al. “Biopsy with Thermally-Responsive Untethered Microtools.” DOI: 10.1002/adma.201203348; Adv. Mater. 2013, 25, 514-519.
Zeng, D. et al. “Optical trepaning using axicon lenses.” doi.org/10.2351/1.5060534; ICALEO® 2005 Congress Proceedings; Laser Microfabrication Conference.
Zeng, D. et al. “Characteristic analysis of a refractive axicon system for optical trepanning”; DOI: 10.1117/1.2353119; Optical Engineering 45(9), 094302, September 2006.
Behnan, J. et al. “Ultrasonic Surgical Aspirate is a Reliable Source For Culturing Glioblastoma Stem Cells”; DOI: 10.1038/srep32788; Scientific Reports; 6:32788.
Vaskova, M. et al. “Cytometric analysis of cell suspension generated by cavitron ultrasonic surgical aspirator in pediatric brain tumors”; doi.org/10.1007/sl1060-019-03135-w; Journal of Neuro-Oncology (2019) 143:15-25.
Zadeh, G. et al. “Diagnostic implications of histological analysis of neurosurgical aspirate in addition to routine resections”; doi:10.11 ll/j.1440-1789.2011.01234.x; Neuropathology 2012; 32, 44-50.
Lei, B. U., et al. “Absorbent Microbiopsy Sampling and RNA Extraction for Minimally Invasive, Simultaneous Blood and Skin Analysis.” J. Vis. Exp. (144), e58614, doi:10.3791/58614 (2019).

1. A tissue excision apparatus comprising:
a laser configured for emission of a beam of electromagnetic energy;
optical components configured to modify the beam of electromagnetic energy to form an annular converging beam; and
a control system configured to limit the emission of the beam of electromagnetic energy to a duration between 10 picoseconds and 10 milliseconds.

2. The tissue excision apparatus of claim 1 wherein the laser is configured to emit the beam of electromagnetic energy at a wavelength between 1.2 μm and 2.5 μm.

3. The tissue excision apparatus of claim 1 or claim 2 wherein the emission of the beam of electromagnetic energy has a pulse energy between 10 mJ and 10 J.

4. The tissue excision apparatus of any one of the preceding claims wherein the laser is a Ho:YAG laser.

5. The tissue excision apparatus of any one of the preceding claims wherein the optical components comprise a non-spherical lens.

6. The tissue excision apparatus of any one of the preceding claims wherein the optical components comprise an axicon.

7. The tissue excision apparatus of any one of the preceding claims wherein the optical components comprise an optical element configured to collimate the beam.

8. The tissue excision apparatus of claim 7 wherein the optical element is a lens.

9. The tissue excision apparatus of any one of the preceding claims wherein the optical components comprise a reflective collimator.

10. The tissue excision apparatus of any one of the preceding claims wherein the beam of electromagnetic energy is directed through an optical fiber.

11. The tissue excision apparatus of claim 10 wherein the optical fiber is a multimode fiber.

12. The tissue excision apparatus of any one of the preceding claims wherein the annular converging beam comprises a peripheral portion and a central portion.

13. The tissue excision apparatus of claim 12 wherein the central portion of the annular converging beam has lower electromagnetic energy fluence than the peripheral portion.

14. The tissue excision apparatus of any one of the preceding claims wherein the tissue excision apparatus further comprises a cooling device configured to direct a coolant toward the beam of electromagnetic energy.

15. The tissue excision apparatus of claim 14 wherein the control system is configured to synchronize an application of the coolant with the emission of the beam of electromagnetic energy.

16. The tissue excision apparatus of claim 14 or 15 wherein the cooling device comprises a solenoid valve.

17. The tissue excision apparatus of any one of claims 14-16 wherein the cooling device comprises a nozzle.

18. The tissue excision apparatus of any one of claims 14-17 wherein the cooling device is configured to direct 1,1,1,2-Tetrafluoroethane or CO₂ toward the beam of electromagnetic energy.

19. A method of excising tissue, the method comprising:
directing a beam of electromagnetic energy to a surface of a tissue, wherein the beam of electromagnetic energy is an annular converging beam; and
displacing a portion of the tissue.

20. The method of claim 19 wherein the beam is an annular converging cone-shaped beam.

21. The method of claim 19 or 20 wherein the beam of electromagnetic energy is directed to the surface in a single pulse with a duration between 10 picoseconds and 10 milliseconds.

22. The method of any one of claims 19-21 wherein the single pulse comprises a pulse energy of between 10 mJ and 10 J.

23. The method of any one of claims 19-21 wherein the electromagnetic energy has a wavelength between 1.2 μm and 2.5 μm.

24. The method of any one of claims 19-23 wherein displacing the portion of the tissue forms a void in the tissue.

25. The method of claim 24 wherein the void extends at least 100 μm from the surface of the tissue.

26. The method of claim 24 or claim 25 wherein the void has a diameter between 100 μm and 1 mm.

27. The method of any one of claims 19-26 wherein the portion of the tissue has a volume between 0.003 mm³ and 0.3 mm³.

28. The method of any one of claims 19-26 wherein the portion of the tissue has a volume between 0.010 mm³ and 0.1 mm³.

29. The method of any one of claims 19-28 further comprising processing the portion of the tissue.

30. The method of claim 30 wherein processing the portion of the tissue comprises performing a diagnostic technique on the portion of the tissue.

31. The method of claim 30 wherein processing the portion of the tissue comprises performing a tissue culture.

32. The method of claim 30 wherein processing the portion of the tissue comprises performing a histopathological examination.

33. The method of claim 30 wherein processing the portion of the tissue comprises genetic profiling.

34. The method of claim 30 wherein processing the portion of the tissue comprises flow cytometry.

35. The method of claim 30 wherein processing the portion of the tissue comprises a proteomic assay.

36. The method of claim 30 wherein processing the portion of the tissue comprises mass spectrometry.

37. The method of any one of claims 19-30 further comprising directing a coolant toward the surface of the tissue.

38. The method of claim 37 wherein directing the coolant toward the surface of the tissue is synchronized with directing the beam of electromagnetic energy to the surface of the tissue.

39. The method of claim 37 wherein the coolant is 1,1,1,2-Tetrafluoroethane or CO₂.

40. The method of any one of claims 19-39 further comprising applying an optical clearing technique to the surface of a tissue reduce tissue scattering.

41. The method of claim 40 wherein the optical clearing technique comprises applying a chemical agent to the surface of the tissue.

42. The method of claim 41 wherein the chemical agent is glycerol.

43. The method of claim 40 wherein the optical clearing technique comprises applying mechanical pressure to the surface of the tissue.
